(19) United States
(12) Patent Application Publication
(10) Pub. No.: US 2005/0080237 A1
(43) Pub. Date: Apr. 14, 2005

(54) Oligomeric molecules and uses thereof

(76) Inventors: David B Weiner, Merion, PA (US); Rafick R Sekaly, Laurent (CA)

Correspondence Address: COZEN O'CONNOR, P.C., 1900 MARKET STREET, PHILADELPHIA, PA, 19103-3508, US

(21) Appl. No.: 10/504,400

(22) PCT Filed: Feb. 11, 2003
(86) PCT No.: PCT/US03/04037

(30) Foreign Application Priority Data
Feb. 11, 2002 (US) 60355925

(51) Int. Cl. C07K 16/46; C07K 14/52; C07K 14/475

(52) U.S. Cl. 530/351; 530/391.1; 530/399

(57) ABSTRACT

The present invention relates to novel oligomers that comprise a plurality of monomers that comprises an active moiety and a scaffold moiety. The oligomers may comprise 6-12 monomers or more. The active moiety comprises sequences that interact with other proteins. The scaffold moiety comprises sequences that interact with sequences of scaffold moieties from other monomers to form oligomers.

### FIELD OF THE INVENTION

The present invention relates to oligomers which comprise an active region and a oligomerizing region.

### BACKGROUND OF THE INVENTION

Current Immune therapy and receptor ligand therapy is hampered by the restriction of a single class of molecules which can be used to target specific receptors. In practice immunoglobulins of the IgG class have served the mainstay as in vivo immune therapy platforms. However these molecules suffer in their limited valency which results in low affinity interactions and poor receptor targeting resulting in nonspecificity. Additionally, immune properties of these molecules in some instances is a drawback.

There is a need for improved molecules that function with the specificity of immunoglobulins but which exhibit a higher affinity and improved targeting. More generally, there is a need for improved biologically active proteins that have improved function over native species.

### SUMMARY OF THE INVENTION

The present invention relates to oligomers that comprise a plurality of monomers that comprises an active moiety and a scaffold moiety. The oligomers may comprise 6-12 monomers or more. The active moiety comprises immunoglobulin sequences that interact with other proteins. The scaffold moiety comprises non-immunoglobulin sequences that interact with non-immunoglobulin sequences of scaffold moieties from other monomers to form oligomers.

Another aspect of the invention relates to oligomers that comprise a plurality of monomers comprising an active moiety and a scaffold moiety. The oligomer comprises 6-12 monomers or more. The active moiety comprises sequences that interact with other proteins. The active moiety comprises at least a protein binding fragment of a protein selected from the group consisting of: cytokines, cytokine receptors, chemokines, chemokine receptors, adhesion molecules, costimulatory molecules, growth factors, growth factor receptors, blood coagulation factors and enzymes. The scaffold moiety comprises sequences that interact with sequences of scaffold moieties from other monomers to form oligomers.

Another aspect of the invention relates to oligomers that comprise a plurality of monomers comprising an active moiety and a scaffold moiety. The oligomer comprises 6-12 monomers or more. The active moiety comprises at least a protein binding fragment of a protein selected from the group consisting of: antibodies, cytokines, cytokine receptors, chemokines, chemokine receptors, adhesion molecules, costimulatory molecules, growth factors and growth factor receptors. The scaffold moiety comprises sequences that interact with sequences of scaffold moieties from other monomers to form oligomers, wherein the scaffold moiety bind to a protein selected from the group consisting of: APAF-1 cytochrome C; N-Ethylmaleimide-sensitive Fusion Protein; Katanin; rotavirus non-structural protein 2; alpha-synuclein; Porphobilinogen Synthase; Catabolic Ornithine carbamovltransferase; Bromoperoxidase; Yeast Arginine methyltransferase; HMT1; Phage φ 29 Histone-like Protein p6; Cadherin; and Ninth Complement Component poly-C9.

The present invention relates to compositions, including pharmaceutical composition, that comprise the oligomers of the invention.

The present invention relates to uses of the oligomers of the invention. The uses relate to the nature of the active moiety. In embodiments in which the active moiety is an immunoglobulin derived sequence which specifically binds to another protein, such as embodiments in which the active moiety is a binding sequence of an antibody, the oligomers may be used in the manner in which the antibodies are used such as in immunoassays, affinity columns and as therapeutics and imaging agents. In embodiments in which the active moiety is sequence which specifically binds to a receptor, such as embodiments in which the active moiety is a binding sequence of an receptor binding ligand, the oligomers may be used in the manner in which the ligands, are used such as in assays, columns and as therapeutics agents. In embodiments in which the active moiety is sequence which specifically binds to a receptor, such as embodiments in which the active moiety is a binding sequence of an receptor, the oligomers may be-used in the manner in which the soluble receptors are used such as in assays, columns and as therapeutics agents. In embodiments in which the active moiety is sequence from a blood coagulation factor, the oligomers may be used in the manner in which the blood coagulation factors are used such as in assays and as therapeutics agents. In embodiments in which the active moiety is an active site of an enzyme, the oligomers may be used in the manner in which the enzymes are used such as in assays, processes and as therapeutics agents. The present invention relates to, methods in which the oligomers are used for such purposes.

### DETAILED DESCRIPTION OF PREFERRED EMBODIMENTS

As used herein, “oligomer” refers to a molecule that comprising at least six subunit monomers. In some embodiments, the oligomer comprises at least 7 monomers. In some embodiments, the oligomer comprises at least 8 monomers. In some embodiments, the oligomer comprises at least 9 monomers. In some embodiments, the oligomer comprises at least 10 monomers. In some embodiments, the oligomer comprises at least 11 monomers. In some embodiments, the oligomer comprises at least 12 ionomers. In some embodiments, the oligomer comprises more than 12 monomers.

The plurality of monomers may be different or the same. In some embodiments, the oligomer is a homo-oligomer comprising identical monomers. In some embodiments, the oligomer is a hetero-oligomer comprising non-identical monomers. In some embodiments, the oligomer is a hetero-oligomer comprising non-identical monomers including at least six identical monomers. In some embodiments, the oligomer is a hetero-oligomer comprising non-identical monomers including at least 7 identical monomers. In some embodiments, the oligomer is a hetero-oligomer comprising non-identical monomers including at least 8 identical monomers. In some embodiments, the oligomer is a hetero-oligomer comprising non-identical monomers including at least 9 identical monomers. In some embodiments, the oligomer is a hetero-oligomer comprising non-identical monomers including at least 10 identical monomers. In some embodiments, the oligomer is a hetero-oligomer comprising non-identical monomers including at, least 11 identical monomers. In some embodiments, the oligomer is a hetero-oligomer comprising non-identical monomers including at least 12 identical monomers. In some embodiments, the oligomer is a hetero-oligomer comprising non-identical monomers including more than 12 identical monomers.

In some embodiments, heterodimers include different monomers which complement the activity of each other. For example, an oligomer may include a monomer with an active moiety from a molecule involved in immunity, such as CD40 ligand, together with a monomer that comprises an active moiety based upon an antigen or MHC/antigen complex sequence in order to achieve an enhanced immune response. Similarly, an oligomer may include a monomer with an active moiety from a molecule such a death signal together with a monomer that comprises an active moiety based upon an antigen or MHC/antigen complex sequence in order to kill cells reactive to the monomer.

In some embodiments, the active moiety comprises immunoglobulin sequences that interact with other proteins. According to some of these embodiments, the active moiety is derived from or functions as an antibody-binding region and the oligomer functions as an improved antibody. The antigenic target of such oligomers may be infectious agents, cancer markers or other cancer targets, messenger molecules, molecules associated with diseases such as autoimmune disease or any other target molecule for which an antibody may function for specific binding. In some embodiments, the oligomer binds to a pathogen protein, a cancer cell, cytokines, cytokine receptors, chemokines, chemokine receptors, adhesion molecules, costimulatory molecules, growth factors and growth factor receptors. Preferred targets include TNF, IL-1, IL-2, IL-1R, toxin associated with septic shock, and rabies virus protein. In some preferred embodiments the active moiety comprises an antibody complementary determining region. According to some of these embodiments, the active moiety comprises an antibody variable region. In embodiments in which the active moiety is an antibody sequence, the scaffold moiety is generally a non-immunoglobulin derived protein.

According to some embodiments, the active moiety comprises receptor sequences which bind to receptor ligands. These oligomers are useful as soluble receptor analogs and can be used to bind to receptor ligands. According to some of these embodiments, the active moiety comprises receptor sequences which bind to receptor ligands wherein the receptor is selected from the group consisting of: cytokine receptors and growth factor receptors. According to some of these embodiments, the active moiety comprises receptor sequences which bind to receptor ligands wherein the receptor is selected from the group consisting of: IL-1 receptor, TNF receptor, and IGF receptors. According to some embodiments, the active moiety comprises at least a protein binding fragment of a protein selected from the group consisting of: cytokines, cytokine receptors, chemokines, chemokine receptors, adhesion molecules, costimulatory molecules, growth factors, growth factor receptors, blood coagulation factors and enzymes. In such embodiments, the oligomer retains the activity of the protein form which the active moiety is derived.

Each monomer comprises a scaffold moiety which aggregates to form multimeric complexes. It some embodiments, the monomers aggregate spontaneously or otherwise self-aggregate. In some embodiments, monomers form aggregates with other monomers by noncovalent bonds. In some embodiments, monomers form aggregates with other monomers by covalent bonds. In some embodiments, the scaffold moiety comprises at least a fragment of protein which forms self aggregates with other identical protein molecules by non-covalent bonds. In some embodiments, the scaffold: moiety is at least a fragment of a protein selected from the group consisting of: APAF-1 cytochrome C; N-Ethylmaleimide-sensitive Fusion Protein; Katanin; rotavirus non-structural protein 2; alpha-synuclein; Porphobilinogen Synthase; Catabolic Omithine carbamovltransferase; Bromoperoxidase; Yeast Arginine methyltransferase; HMT1; Phage φ 29 Histone-like Protein p6; Cadherin; and Ninth Complement Component poly-C9.

Each of the patents, patent applications, and publications described herein is hereby incorporated by reference in its entirety.

Various modifications of the invention, in addition, to those described herein, will be apparent to those of skill in the art in view of the foregoing description. Such modifications are also intended to fall within the scope of the appended claims. The present invention is further demonstrated in the following examples that are for purposes of illustration and are not intended to limit the scope of the present invention.

### EXAMPLES

### Example 1

The invention arises from the recognition that it would represent a significant advantage to have molecules that targeted ligands more specifically through enhanced binding properties. In addition it would be a specific advantage to have molecules that have different properties that do not impose directly an immune phenotype. For example developing molecule scaffolds that are not divalent but heptavalent would improve specificity, and targeting ability in vivo at least 4 fold. A dodecamer would increase specificity and targeting at least 5×. Therefore we have focused on developing novel molecules which naturally aggregate to form ordered higher molecular structures. These scaffolds now form the basis of a new generation of molecules that can target any structure that is available on the host cells. Complement component C9 can be engineered to lack hemolytic activity and this would be part of this invention. However this molecule is still able to form dodecamers. Thus this molecule can now be fused through standard molecular biology approaches to cytokines or other ligands that target specific molecules or pieces of antibody Fc portions to not target a ligand in vivo. The new targeted ligand will be part of the C9 dodecamer and thus possess enhanced ligand specificity and targeting ability. Carbohydrate binding domains as well as coupling to allow for nucleic acid binding is also envisioned. As one example a fusion between the C9 scaffold and the TNF-receptor external domain or a FAB that targets TNFα, would result in a molecule that neutralizes TNFα in vivo 5× more efficiently than currently available approaches. The current approaches are all IgG fusions including as Remicade or Embrel.

Several examples of additional multimeric molecules which can similarly be used such as fragments of these molecules that similarly aggregate can also be used in a similar fashion. Examples include APAF-1, Oligomeric NSF ATP oligomerized Katanin, Octameric NSP2, Activated Alpha-synuclein, Porphobilinogen Synthase, OTC, Bromoperoxidase, HMT1, Phage p6, and Cadherin, among others. These fusion molecules have the capacity to significantly improve in vivo therapeutic targeting.

### Example 2

Cytokines useful to produce oligomers according to the present invention include the following.

Tumor Necrosis Factor-alpha, (TNF-alpha) (Nedwin et al., Nucleic Acids. Res. 13:6361-6373).

Tumor necrosis factor-beta. (TNF-beta) (Nedwin et al, Nucleic Acids. Res. 13:6361-6373).

IL-1, (alpha and beta subunits) Genbank Acc. Nos. X03833 and X04500, respectively.

IL-2, Genbank Acc. No. S82692

IL-3, Genbank Acc. No. K01668

IL-4, Arai, et al., J. Immunol. (1989) 142:274-282.

IL-5, Campbell et al., Proc. Natl. Acad. Sci. USA (1987) 84:6629-6633.

IL-6, Genbank Ace. No. J03783

IL-7, Genbank Acc. No. J04156

IL-8, Genbank Acc. No. Y00787

IL-9, Genbank Acc. No. S63356

IL-10, Genbank Acc. No. BC022315

IL-11, Genbank Acc. No. U03421

IL-12B, Genbank Acc. No. M65272

Interferon-alpha. (IFN-alpha), Pestka, (1981) in Methods in Immunol., Vol. 78, Academic Press, NY.

Interferon-beta (IFN-beta), Taniguchi et al, Gene (1980) 10:11-15

Interferon-gamma. (IFN-gamma), Gray and Goeddel, Nature (1982)298:859-863.

Macrophage Chemotactic Protein-1 (MCP-1). Yoshimura, et al., FEBS Lett. (1989) 244:487-493

MAP kinase, Genbank Acc. No. U66243

Leukemia Inhibition Factor, Gough et al., Proc. Natl. Acad. Sci. USA (1988) 85:2623-2627

Erythropoetin, Jacobs et al., Nature (1985) 313:806-810.

Granulocyte Macrophage Colony Stimulating Factor (GM-CSF), Miyatake, et al., EMBO J. (1985) 4:2561-2568.

Granulocyte Colony Stimulating factor (G-CSF), Nagata, BioEssays (1989) 10:113-117.

Lymphotactin (LTN) or Chemokine-C, Genbank Acc. No. D43768

Oncostatin, Genbank Acc. No. AF129855

Amphiregulin, Genbank Acc. No. D12648

Mullerian Inhibiting Substance, Genbank Acc. No. S98336

B-Cell Growth Factor, Lernhardt, et al., Lernhardt, et al., Curr. Topics Microbiol. Immunol. (1986) 132:98-104.

Stem Cell Factor, Genbank Acc. No. AB009245

Mammastatin, Ogawa, et al., J. Chem. Soc. Chem. Commun. (1991) 13:890-891.

### Example 3

Costimulatory molecules useful to produce oligomers according to the present invention include the following:
B7, (CD80), Genbank Acc. No. U47924
RANTES, (Chemokine ligand 2), Genbank Acc. No. AAM54046
B7-1, Genbank Acc. No. AH002809
B7-2, Genbank Acc. No. U04343
B7-3
B7-4
CD40
CD40Ligand.

### Example 4

There are two classes of chemokines, C—X—C (alpha) and C—C (beta), depending on whether the first two cysteines are separated by a single amino acid (C—X—C) or are adjacent (C—C). The alpha-chemokines, such as interleukin-8 (IL-8), neutrophil-activating protein-2 (NAP-2) and melanoma growth stimulatory activity protein (MGSA) are chemotactic primarily for neutrophils, whereas beta-chemokines, such as RANTES, MIP-1alpha, MIP-1beta, monocyte chemotactic protein-i (MCP-1), MCP-2, MCP-3 and eotaxin are chemotactic for macrophages, T-cells, eosinophils and basophils (Deng, et al., Nature, 381, 661-666 (1996)).

The chemokines bind specific cell-surface receptors belonging to the family of G-protein coupled seven-transmembrane-domain proteins (reviewed in Horuk, Trends Pharm. Sci., 15, 159-165 (1994)) which are termed “chemokine receptors.”

Macrophage Migration Inhibitory Factor, Genbank Acc. No. L19686

Platelet Factor 4, Genbank Acc. No. M25898

Connective Tissue Activating. Protein III, (beta-thromboglobulin), Brant, et al., J. Leucocyte Biol. (2000)67:471-478.

The beta-chemokines include eotaxins, MIP (“macrophage inflammatory protein”), MCP (“monocyte chemoattractant protein”) and RANTES (“regulation-upon-activation, normal T expressed and secreted”).

Exemplary chemokines include interleukin-8, dendritic cell chemokine 1. (DC-CK1) and lymphotactin, which is a chemokine important for recruitment of gamma and delta. T cells and for mucosal immunity, as well as other members of the C—C and C—X—C chemokine subfamilies (see, for example, Miller and Krangel, Crit. Rev. Immunol. 12:17-46 (1992); Schall, “The Chemokines” in Thomson, (1994) Hedrick et al., J. Immunol. 1588:1533-1540 (1997); and Boismenu et al., J. Immunol. 157:985-992 (1996), each of which are incorporated herein by reference).

GRO.alpha, Wood and Richmond, J. Biol. Chem. (1995)270:30619-30626.

GRO.beta, and GRO.gamma Zagorski and Delarco, Protein Express. Purif. (1994) 5:337-345.

### Example 5

A variety of diffusible factors which stimulate the growth of cells in a hormone-like manner are generally called “growth factors”. Growth factors are often present in serum and have also been isolated from a variety of organs. They are protein molecules (or groups of such molecules) and in all known cases they interact with specific cell surface receptors to promote cellular growth and/or differentiation. Growth factors vary in their tissue specificity, i.e. some interact only with specific cell types, while others are active on a wider cell type range. Some of those shown below are in reality “families” of related proteins, e.g., EGF, FGF and nerve growth factor.
Platelet Derived Growth Factor (PDGF) or PDGF-alpha, Genbank Acc. No. AH002927
Epidermal Growth Factor (EGF), or low-density lipoprotein receptor, represented by Genbank Acc. No. AF023155
Basic Fibroblast Growth Factor, Florkiewicz and Sommer, Proc. Natl. Acad. Sci. USA 86:3978-3981.
Acidic Fibroblast Growth Factor, Genbank Acc. No. BG706412
Vascular Endothelial Growth Factor (VEGF). Genbank Acc. No. AF022375
Nerve Growth Factor or neurotrophic growth factor, represented by Genbank Acc. No. AH001904
Insulin-Like Growth Factor (somatomedin, relaxin), Genbank Acc. No. X57025.

### Example 6

Some CAMS and Integrins useful to produce active moieties according to the invention include:
PECAM-1, (CD31), Genbank Ace. No. BC022512
ICAM-1, Genbank Acc. No. J03132
VCAM, Genbank Acc. No. AU121762
E-Selectin (ELAM)—Genbank Acc. No. M24736
L-Selectin (lymphocyte adhesion molecule), Genbank Acc. No. AL021940
P-Selectin (platelet selectin), Genbank Acc. No. L01574
Vitronectin, Genbank Acc. No. A1352496
alpha_{X}beta_{Y} platelet integrins, of which alpha_{V}beta₃ is representative and most directly applicable to the inflammatory cascade of atherosclerosis (Yamani et al., J. Amer. Coll. Cardiol. (2002) 39:804-810.

Further adhesion molecules are discussed in the Adhesion Molecule Facts Book, by Clare Isacke, Published: June 2000, Publisher: Academic Press, Incorporated, Edition: 2nd Edition, ISBN: 0123565057, which is incorporated by reference in its entirety.

### Example 7

An example of a blood coagulation factor useful to produce an active moiety is Factor VIII, Genbank Acc. No. M14113.

### Example 8

Examples of multimeric respiratory proteases are alphaketoglutrate dehydrogenase, aspartate transcarbamylase, and cytochrome oxidase.

### Example 9

Examples of proteases useful to produce an active moiety are Pepsin, Cathepsins, Papain, Trypsin, Chymotrypsin, Carboxypeptidase and Thyroxine Binding Globulin (serine protease inhibitor)—Genbank Acc. No. M14091.

### Example 10

The following are examples of multimetric molecules which can be used to prepare scaffold moieties.
1. APAF-1 CYTOCHROME C multimeric complex
   (J Biol Chem, Vol274:11549-11556,1999.)
   APAF-1: important apoptosis activator,
   Protein: 130 kDa, 1194aa's.
   Recombinant APAF-1 in Baculovirus expression system.
   Both dATP and Cytochrome c are required to form the multimeric complex.
   The complex contains at least 8 subunits of APAF-I (>1.3 million Da).
   APAF-1-AF013263
2. Oligomeric NSF: N-Ethylmaleimide-sensitive Fusion Protein.
   (J Biol Chem, Vol. 273:15675-15681,1998.)
   NSF is an ATPase, involved in intracellular Mb trafficking.
   hexagonal cylinder in solution (>90%)+trimer & dodecamer.
   Each 85 kDa NSF subunit contains 3 primary domains:
   Amino terminal N-domain required for substrate binding,
   2 ATPase domains, D1I (for membrane transport activity) and D2 (for oligomerization).
3. NSF hexamer is held together by oligomerization of its D2 domains.
   Produced in E. coli.
   Equilibrium Sedimentation experiments.
   Presence of ATF essential for hexamer formation and/or stabilization.
   510 kDa: hexameric molecular mass of His6NSF-Myc.
   EM: 13×10 nm hollow cylinder with a larger Surface Area and increased subunit flexibility vs. that expected for a sphere of same mass and density.
   NSF-AF135168
4. ATP-dependent oligomerization of Katanin.
   (Science, vol. 286:782-785,1999.)
   Katanin: member of AAA ATPase.
   Katanin: heterodimer organized into a 60 kD enzymatic subunit (p60) and a targeting subunit p80)
   p60 subunit of Katanin oligomerizes in an ATP-dependent manner.
   Might be unstable, and in equilibrium with monomers.
   Katanin—(2 subunits) AF056022
5. Octameric NSP2 (rotavirus non-structural protein2).
   (Schuck P., et al. J. Biol Chem, 276(13):9679.2001.)
   rotavirus NS protein involved in formation of viroplasm and synthesis of dsRNA in vivo+NTPase activity.
   NSP2: 35 kDa protein that forms octamers (301 kDa) in solution.
   studied secondary structure, oligomeric state and hydrodynamic shape of the recombinant protein.
   NSP2 self-assembles into highly stable octamers (based on sedimentation equilibrium, sedimentation velocity and light scattering experiments) under varying conditions of pH and NaCl.
   the free NSP2 monomer concentration is below the sensitivity of the interference optical systems (>0.1 uM).
   NSP2 octamers dissociate in the presence of Mg, but this effect is reversible. (partial dissociation of octamer into tetramer).
   structure: contains a high amount of beta-sheet;
   preliminary pictures from cryo-EM reconstitutions: barrel-shaped particle. NSP2-U92715
6. CopperII-induced self-oligomerization of alpha-synuclein.
   (Peik, S. R., et al. Biochem J., 340:821.1999.)
   alpha synuclein: component of abnormal ptn depositions in senile plaques Alzheimer's and Lewy bodies (Parkinson's); provides possible nucleation center for plaque formation.
   140AA.
   Primary structure divided into 3 regions:
      N-terminal segment (residues 1-60) containing 5-7 KTKEGV repeats,
      Hydrophobic region (61-95), tendency of beta-pleated sheet formation.
      Acidic C-terminus, enriched with acidic AA and proline residues.
   Conformational analysis of recombinant protein: elongated “natively unfolded” structure that could cause the protein to be readily involved in various protein-protein interactions.
   alpha synuclein undergoes self-oligomerization in the presence of copper and zinc (pH dependent).
   Alpha-synuclein-AF007758
7. Porphobilinogen Synthase Octamer.
   (Frankenberg, N., et al. J. Mol. Biol. 289:591.1999.)
   also known as 5-aminolevulinic acid dehydratase
   functions in the first step of tetrapyrrole biosynthesis (e.g. heme & chlorophyll).
   highly conserved among species (bacteria, yeast, human)
   biological unit: homo-octamer.
   MWt: 280 kD
   the 8 subunits are organized as four functional units each composed of a dimer of the protein.
   recombinant soluble human PBGS is produced from an artificial gene in E. coli. (Jaffe. E. K., et al. J. Biol. Chem. 275(4):2619.2000.)
   crystal structure exists.
   Porphobilinogen synthase—BC019323
8. Catabolic Ornithine carbamovltransferase (OCTase) Dodecamer
   (Villeret, V. et al. PNAS 95:2801.1998.)
   (Villeret, V. et al. PNAS 92:10762.1995)
   bacterial enzyme involved in the arginine biosynthetic pathway.
   anabolic OCTase is a trimer of identical 34 kDa subunits.
   catabolic OCTase is a 456 kDa Ptn composed of 4 trimers disposed in a tetrahedral manner (dodecamer).
   recombinant protein overexpressed in yeast model.
   crystal structure exists.
   OCTase—M11266
9. Bromoperoxidase dodecamer.
   (Isupov. M. N. et al. J Mol Biol 299:1305-2000).
   vanadium dependent bromoperoxidase from red algae (VBPO).
   homododecamer, MWt 740 kDa; 64 kDa subunits arranged with 23 cubic point group symmetry.
   each of the cubic faces is made up of a dimer.
   crystal structure exists.
10. Yeast Arginine methyltransferase, HMT1 hexamer
   (Weiss, V. H., et al. Nature Structural Biology. 7-1165.2000.)
   has a human counterpart PRMT-protein arginine methyltraasferase). (Rho, J., et al. J Biol Chem 276:11393.2001).
   hexamer with 3 large negatively charged cavities on its outer surface, each cavity formed by a dimer.
   monomer consists of:
      N-terminal region
      C-terminal domain (the body) is an elongated 9-stranded beta-barrel
      loop that connects the first 2 strands of this beta-barrel includes a rigid V-shaped helix-turn-helix motif (the antenna) involved in dimerization.
   the association of dimers to form hexamers is mediated by hydrophilic interactions.
   crystal structure exists.
11. Phage φ 29 Histone-like Protein p6.
   (Abril, A. M., et al. J Mol Biol 292:581. 1999)
   protein p6 of Bacillus subtilis phage: viral histone-like protein, involved in activation of initiation of DNA replication, early promoter repression and late promoter activation.
   103 aa residues.
   abundant, 6.6×105 copies/cell.
   forms a multimeric protein p6 core for DNA wrapping.
   p6 oligomers larger than hexamers (71 kDa) detected by cross linking with glutaraldehyde in the absence of DNA.
   transmission E-microscopy and image processing: crooked shaped structures that could grow either into doughnut-shaped or filamentous (n-mer) structures.
12. The cadherin zipper model.
   (Shapiro, L. et al. Nature. 374:327.1995)
   (Alattia, J R, et al. FEBS Letters. 4.17; 405.1997)
   cadherins are Ca2+-dependent cell adhesion molecules (CAM) involved in homophilic cell to cell association.
   a cadherin molecule, (e.g. E-cadherin, 120 kDa) is a, single pass transmembrane glycoprotein consisting of:
      five extracellular tandem repeats (cadherin domains, 110aa each),
      a single transmembrane region,
      and a single conserved cytoplasmic domain linked to the cytoskeleton actin filaments via alpha and beta-catenins.
   the cadherin domains have two dimer interfaces that combine to form a zipper-like supermolecular ribbon through the CAD1 domain Ca ions are essential in the stabilization of the structure.

Note: Tomschy et al (EMBO, 15:3507.1996) proposed the ECADCOMP system in which the E-cadherin ectodomain: was fused recombinantly to the coiled-coil assembly domain, of cartilage oligomeric matrix protein (COMP).

This artificially clustered and, soluble E-cadherin is able to self-associate whereas the ectodomain alone is not. Electron Microscopy studies reveal that pentameric ECADCOMP show ring-like structures by interaction of their N-terminal domains to form lateral cis dimers. Two cis-dimers from different ECADCOMP molecules can also interact to form “associated rings” (Pertz O., et al., EMBO J.18:1738.1999).
Crystal structure of the cadherin domain exists.
Cadherins—AK00811
13. Ninth Complement Component poly-C9
   (Podack, E. R., Tschop, J. PNAS. 79:574.1982)
   (Esser, A. F., et. al. Mol Immunol. 33:725.1.996)
   (Tomlinson, S., et al. J Immuno. 155:436.1995)
C9 is the last protein that binds to the assembling complement membrane attack complex.
   C9 is a single chain plasma glycoprotein of 538aa.
   tubular C9 dodecameric polymers form spontaneously in Veronal-buffered saline. (by Electron Microscopy)
   C9 from horse has the characteristic of lacking hemolytic activity, although its structure is similar to that of human C9.
   Complement C—Y08545.

### Example 11

Active moieties of the present invention can mimic binding of antibodies that bind to many molecules including but not limited to those set forth below. It is to be understood that reference to an antigen includes reference to the receptor. For example, “CD20” includes the CD20 antigen as well as the CD20 receptor.

| | |
|---|---|
| Interleukin 1 | Respiratory Syncytial virus (RSV) |
| Interleukin 2 (CD25) | CD3 |
| Interleukin 3 | CD4 |
| Interleukin 4 | CD11a |
| Interleukin 5 | CD18 |
| Interleukin 6 | CD20 |
| Interleukin 7 | CD33 |
| Interleukin 8 | CD40 |
| Interleukin 9 | CD52 |
| Interleukin 10 | CD80 |
| Interleukin 11 | CD95 |
| Interleukin 13 | p53 |
| Interleukin 15 | HER2/neu |
| Interleukin 16 | ERBB3/Her-3 |
| Interleukin-17 | VLA-4 |
| Interleukin-22 | VEGF |
| Tumour necrosis factor (TNF) | IgE |
| Insulin Like Growth Factor I | DcR3/TR6 |
| Platelet Derived Growth Factor | TRICK2 |
| Epidermal Growth Factor | Selectins |
| Transforming Growth Factor | Integrins (alpha and beta) |
| Fibroblast Growth Factor | Cadherins |
| Nerve Growth Factor | ICAM |
| Endothelin | ECAM |
| Human Growth Hormone | PECAM |
| Growth Factor | NCAM |
| Somatotrophin | JAM |
| Erythropoeitin | T-CADHERIN ADHESION |
| tPA | MOLECULE |
| Insulin | SynCAM |
| Factor VII | glycoprotein IIb/IIIa |
| Factor VIII | vitronectin |
| TRAIL | fibronectin |
| TNF | Interferon alpha |
| Fas | Interferon Beta |
| DcR1 | Interferon Gamma |
| DcR2 | GM-CSF |
| OPG | MCP-1 (MCAF) |
| ODF | LIF (leukemia Inhibitory Factor) |
| CLAN | MCP-2 |
| PERP | MCP-3 |
| DEDD2 | PF-4 |
| DR5 | KGF |
| RANK | RANTES |
| DcR1 | Stem Cell Factor (SCF) |
| DcR2 | MIP-1 |

1. An oligomer comprising a plurality of monomers comprising an active moiety and an backbone moiety wherein:
the oligomer comprises at least 6 monomers;
the active moiety comprises immunoglobulin sequences that interact with other proteins; and
the scaffold moiety comprises non-immunoglobulin sequences that interact with non-immunoglobulin sequences of scaffold moieties from other monomers to form oligomers.

2. The oligomer of claim 1 comprising 7-12 monomers.

3. The oligomer of claim 1 wherein the active moiety comprises an antibody complementary determining region.

4. The oligomer of claim 1 wherein the active moiety comprises an antibody variable region

5. The oligomer of claim 1 wherein the active moiety comprises antigen-binding antibody sequences which bind to a protein selected from the group consisting of: cytokines, cytokine receptors, chemokines, chemokine receptors, adhesion molecules, costimulatory molecules, growth factors, growth factor, receptors, blood coagulation factors and enzymes

6. The oligomer of claim 11 wherein the active moiety comprises receptor sequences which bind to receptor ligands.

7. The oligomer of claim 1 wherein the active moiety comprises receptor sequences which bind to receptor ligands wherein the receptor is selected from the group consisting of: cytokine receptors and growth factor receptors.

8. The oligomer of claim 1 wherein the active moiety comprises receptor ligand sequences which bind to receptors.

9. The oligomer of claim 1 wherein the active moiety comprises receptor ligand sequences which bind to receptors wherein the receptor ligand is selected from the group consisting of: cytokines and growth factors.

10. The oligomer of claim 1 wherein the scaffold moiety comprises at least a fragment of monomer which forms aggregates with other monomers by non-covalent bonds.

11. The oligomer of claim 1 wherein the scaffold moiety comprises at least a fragment of a protein which forms self aggregates with other identical protein molecules by non-covalent bonds wherein the scaffold moiety bind to a protein selected from the group consisting of: APAF-1 cytochrome C; N-Ethylmaleimide-sensitive Fusion Protein; Katanin; rotavirus non-structural protein 2; alpha-synuclein; Porphobilinogen Synthase; Catabolic Ornithine carbamovltransferase; Bromoperoxidase; Yeast Arginine methyltransferase; HMT1; Phage φ 29 Histone-like Protein p6; Cadherin; and Ninth Complement Component poly-C9.

12. An oligomer comprising a plurality of monomers comprising an active moiety and a backbone moiety, wherein:
the oligomer comprises at least 6 monomers;
the active moiety comprises sequences that interact with other proteins, said active moiety comprising at least a protein binding fragment of a protein selected from the group consisting of: cytokines, cytokine receptors, chemokines, chemokine receptors, adhesion molecules, costimulatory molecules, growth factors and growth factor receptors; and
the scaffold moiety comprises sequences that interact with sequences of scaffold moieties from other monomers to form oligomers.

13. The oligomer of claim 12 comprising 7-12 monomers.

14. The oligomer of claim 12 wherein the active moiety comprises protein binding sequences from a cytokine.

15. The oligomer of claim 12 wherein the active moiety comprises protein binding sequences from a cytokine receptor.

16. The oligomer of claim 12 wherein the active moiety comprises protein binding sequences from a chemokine.

17. The oligomer of claim 12 wherein the active moiety comprises protein binding sequences from a chemokine receptor.

18. The oligomer of claim 12 wherein the active moiety comprises protein binding sequences from an adhesion molecule.

19. The oligomer of claim 12 wherein the active moiety comprises protein binding sequences from a costimulatory molecule.

20. The oligomer of claim 12 wherein the active moiety comprises protein binding sequences from a growth factor.

21. The oligomer of claim 12 wherein the active moiety comprises protein binding sequences from a growth factor receptor.

22. The oligomer of claim 12 wherein the scaffold moiety comprises at least a fragment of monomer which forms aggregates with other monomers by non-covalent bonds.

23. The oligomer of claim 12 wherein the scaffold moiety comprises at least a fragment of protein which forms self aggregates with other identical protein molecules by non-covalent bonds wherein the scaffold moiety bind to a protein selected from the group consisting of: APAF-1 cytochrome C; N-Ethylmaleimide-sensitive Fusion Protein; Katanin; rotavirus non-structural protein 2; alpha-synuclein; Porphobilinogen Synthase; Catabolic Omithine carbamovltransferase; Bromoperoxidase; Yeast Arginine methyltransferase; HMT1; Phage (p 29 Histone-like Protein p6; Cadherin; and Ninth Complement Component poly-C9.

24. An oligomer comprising a plurality of monomers comprising an active moiety and an backbone moiety, wherein:
the oligomer comprises at least 6 monomers;
the active moiety comprises at least a protein binding fragment of a protein selected from the group consisting of: antibodies, cytokines, cytokine receptors, chemokines, chemokine receptors, adhesion molecules, costimulatory molecules, growth factors and growth factor receptors; and
the scaffold moiety comprises sequences that interact with sequences of scaffold moieties from other monomers to form oligomers, wherein the scaffold moiety bind to a protein selected from the group consisting of: APAF-1 cytochrome C; N-Ethylmaleimide-sensitive Fusion Protein; Katanin; rotavirus non-structural protein 2; alpha-synuclein; Porphobilinogen Synthase; Catabolic Ornithine carbamovltransferase; Bromoperoxidase; Yeast Arginine methyltransferase; HMT1; Phage φ 29 Histone-like Protein p6; Cadherin; and Ninth Complement Component poly-C9.

25. The oligomer of claim 24 comprising 7-12 monomers.
